(19) United States
(12) Patent Application Publication
(10) Pub. No.: US 2007/0141576 A1
(43) Pub. Date: Jun. 21, 2007

(54) Biological chip and use thereof

(75) Inventor: Masafumi Koide, Aichi (JP)

Correspondence Address: YOUNG & THOMPSON, 745 SOUTH 23RD STREET, 2ND FLOOR, ARLINGTON, VA, 22202, US

(73) Assignee: BBK BIO CORPORPATION, Tokyo (JP)

(21) Appl. No.: 10/582,526

(22) PCT Filed: Dec. 13, 2004
(86) PCT No.: PCT/JP04/18580

§ 371 Date: Jun. 9, 2006

(51) Int. Cl. C12Q 1/68; C12M 3/00

(52) U.S. Cl. 435/6; 435/287.2; 977/924

(57) ABSTRACT

A biological chip includes probes immobilized on a circumferential side wall of a discoidal or cylindrical substrate. The probes contain a nucleic acid, a peptide, a saccharide, a lipid, a chemical substance, or a fragment thereof. The disc or cylinder is rotated so as to attain rapid intake of massive assay information obtained from plural probe groups arranged on the circumferential side wall. Mass production of a lot of chips or large-capacity chips is accomplished by constructing the disc or cylinder from a large number of thin sheets or through lamination of plural thin sheets. In blood tests, a biological chip having incubation cells arranged thereon realizes system simplification and miniaturization and resource savings.

### TECHNICAL FIELD

The present invention relates to biological chips and use thereof, which clarify origins and patterns of manifestation of various vital phenomena and reveal, for example, genotypes, gene expression levels, relations between ligands and receptors, enzymatic activities, and biological levels of materials.

### Background Art

Genomic, proteomic, and transcriptomic analyses of organisms have been increasingly conducted in medical and biotechnological fields. Vital activities are diverse and complicated phenomena that cannot be expressed as a simple combination of chemical reactions. However, a variety of and a large quantity of molecular information must be obtained so as to come closest to the actual natures of such vital phenomena.

Accordingly, biological chips and assay systems therefor have been practically used. The biological chips include DNA chips and protein chips for evaluating the sequence and expression of genes in a collective manner. An example of these biological chips can be found in U.S. Pat. No. 5,744,305.

Regular DNA chips each comprise a plate-like substrate and oligonucleotides having predetermined nucleotide sequences as one of DNA probes, in which the oligonucleotides are immobilized as spots at appropriate intervals on the substrate.

The substrate (plate-like substrate) includes, for example, glass substrates, silicon substrates, and plastic substrates. Among them, slide-glass substrates for microscopic use are most widely used.

The probe spots are arranged as a matrix on a plane at intervals of 50 μm to 500 μm in one DNA chip. The oligonucleotides contained in the spots have nucleotide sequences varying from one spot to another.

Methods for arranging oligonucleotides on a substrate include a method in which oligonucleotides are immobilized on the substrate while elongating the oligonucleotides by one base (one nucleotide); and a method in which oligonucleotides have been synthesized in advance and are then immobilized on the substrate. The former method is representatively carried out by a photolithography technique. The latter method is carried out, for example, by a mechanical micro-spotting technique. An ink-jet process can be applied to any of the two methods.

The photolithography technique uses a photo-reactive protecting group (see Fordor et al., Science, 251, 767 (1991)). The formation of spots (probes) according to the photolithography technique will be illustrated below.

Initially, an amino group having the photo-reactive protecting group is immobilized overall the substrate. Next, light is selectively applied to a target spot to which a desired base will be bound according to a photolithography technique used in regular semiconductor production processes. In the spot irradiated with the light, the bond between the photo-reactive protecting group and the amino group has been cleaved, and the amino group alone remains. The desired base having the same protecting group at the end is bound to the amino group. Next, using a photomask having another shape, light is selectively applied to another target spot. A base having the protecting group is bound thereto by the same procedure as above. The process of elongating an oligonucleotide by one base is repeated until desired nucleotide sequences are obtained in all the spots. Thus, different oligonucleotides are arrayed on the substrate.

The ink-jet process is a technique in which very small droplets are ejected to a predetermined position on a two-dimensional plane using heat or piezoelectric effect.

DNA chips are produced by the ink-jet process in the following manner. Upon application of a voltage to a piezoelectric element connected to a liquid chamber, the piezoelectric element changes its volume, which causes a liquid in the chamber to be ejected as a droplet from a capillary connected to the chamber. The size of the droplet to be ejected is determined by the diameter of the capillary, the change in volume of the piezoelectric element, and the physical properties of the liquid.

DNA chip production units using an ink-jet device are so configured as to enable a desired droplet, such as a certain base, to be applied to a desired spot on a DNA chip by the action of relative movement between the ink-jet device and the DNA chip substrate. Such DNA chip production units using an ink-jet device are roughly categorized as two types. One is a DNA chip production unit using a single-head ink-jet device, and the other is a DNA chip production unit using a multi-head ink-jet device.

The DNA chip production unit using a single-head ink-jet device is so configured as to add a reagent dropwise to a desired spot for removing the terminal protecting group of an oligomer. The protecting group of the spot to which a desired base is to be introduced is removed in this manner to thereby activate the oligomer, and a reaction for binding the desired base is carried out overall the DNA chip. In this procedure, the desired base is bound only to the spot comprising the oligomer having an activated terminal, which terminal has been activated by the action of the reagent added dropwise from the ink-jet device. Then, the terminal of the newly added base is protected. The above procedure is repeated on another spot from which the protecting group is to be removed, until a desired nucleotide sequence is obtained.

The DNA chip production unit using a multi-head ink-jet device is so configured as to provide one ink-jet head per reagent containing a base to thereby enable the direct binding of a desired base with a desired spot. This yields a higher throughput than the DNA chip production unit using a single-head ink-jet device.

Of the techniques for immobilizing a previously prepared oligonucleotide on a substrate, the mechanical micro-spotting technique is a technique in which a liquid containing an oligonucleotide attached at the tip of a stainless steel pin is mechanically pressed on a substrate to thereby immobilize the oligonucleotide thereon. After micro-spotting, an aftertreatment such as immobilization upon irradiation with ultraviolet rays is conducted.

The nucleotide sequence of a target site of a test object is determined using a thus-prepared DNA chip in the following manner.

A sample DNA or RNA is added to a DNA chip carrying an immobilized oligonucleotide probe, as is described above. The sample DNA or RNA has been amplified and labeled with a phosphor.

When the sequence of a DNA in the sample is complementary (in correspondence between adenine and thymine or between cytosine and guanine) with the sequence of the oligonucleotide immobilized on the DNA chip and when the two components are in a liquid phase, a partial duplex derived from hydrogen bonds is formed between bases in the sample and corresponding bases on the chip. Specifically, hybridization occurs.

After the hybridization, uncaptured DNAs are removed from the sample by rinsing. As a result, only the DNA having a complementary sequence with the probe oligonucleotide emits fluorescence at the position where the probe is immobilized. A radioactive isotope is also used for labeling instead of or in addition to the phosphor.

The result of hybridization is quantitatively determined by measuring the fluorescence intensities of regions where the respective probes reside using an array reader. Two main types of such array readers categorized by the assay system are a system in which the fluorescence intensities of plural spots are measured simultaneously using a CCD camera; and a system in which two-dimensional scanning is carried out on the biological chip using a confocal laser microscope. The confocal laser microscope uses, for example, a photomultiplier or an avalanche photodiode as the photodetector.

A dichroic mirror for wavelength separation or a beam splitter for spatial separation of the exciting light and the fluorescence is used, because the fluorescence emitted from the phosphor coupled with the sample has an intensity significantly lower than the intensity of the applied exciting light. The assay of fluorescence intensity does not require intensity calibration and is generally used at two or more wavelengths. In the system using a CCD camera, a large area is excited at once using a lamp as the exciting light source. In the system using a confocal laser microscope, laser is used as the exciting light source, and only the inside region of the focused laser spot is excited.

Accordingly, the assay of a large area of about 1 square centimeter can be conducted in one step according to the system using a CCD camera. In contrast, the assay of a small area with a diameter about 5 to about 30 μm is conducted in one step according to the system using a confocal laser microscope. In addition, the confocal system shows a lower throughput upon read out, because it requires the two-dimensional scanning of the DNA chip or microscope head. The two-dimensional scanning is carried out by moving the stage bearing the substrate in two directions (the X- and Y-directions). When an area 20 mm wide and 60 mm long is scanned at a resolution of 10 μm, it typically takes about five to about fifteen minutes to carry out the scanning.

However, the confocal system can yield a higher S/N ratio than the CCD system, because noise rays and flares can be removed by the stop down of the depth of fluorescence emission.

The distribution of measured fluorescence intensities on the DNA chip is outputted as a two-dimensional image data having an amount of information of about 16 bits.

Artificial coloring can be conducted for easier image analysis. A high fluorescence intensity of a spot in the outputted image indicates that the sample contains a large amount of a cDNA sequence complementary with the sequence of the oligonucleotide in the spot.

Consequently, a gene group expressed in a large quantity in the sample can be identified, and the nucleotide sequence of a target gene can be determined by determining which spot shows a high fluorescence intensity.

Attempts have been made to apply protein chips to various models such as the receptor-ligand relationship, antigen-antibody relationship, enzyme-substrate relationship, and agent-molecular point of action relationship. In many of these attempts, the probes are arranged two-dimensionally on a plane or are spread one-dimensionally, and their reaction conditions, complicated measuring procedures, and evaluation methods are still susceptible to improvements.

Assay systems in the field of blood biochemistry frequently include all the mixing system for mixing the sample and the reagent using an automatic sampler, the conveying system by the action of line control, and the assay system. Accordingly, these assays require a large amount of the blood, expensive and complicated instruments, and large amounts of reagents, and yield large amounts of wastes.

### PROBLEMS TO BE SOLVED BY THE INVENTION

The conventional biological chips require enormous amounts of resource, effort, and time for their productions, because a matrix structure two-dimensionally spread on a plane must be formed, from which chips must be basically prepared by one chip. In addition, analysis systems requiring the same procedures cannot significantly carry out assays with high precision at high speed. Accordingly, an object of the present application (present invention) is to provide a biological chip that can be produced in quantities at low cost and enables high-performance and high-throughput assays and analyses. Another object of the present invention is to provide a system using the biological chip. Yet another object of the present invention is to provide a novel hematological testing system using the biological chip from the viewpoints of saving cost and energy, because conventional autoanalyzers used in blood tests are expensive, inevitably suffer a breakdown and consume large amounts of reagents.

### DISCLOSURE OF INVENTION

The present invention provides, in a first aspect, a biological chip comprising a substrate; and a probe as a biologic material or artificial biologic material immobilized on a surface of the substrate, in which the substrate is in the form of a column or cylinder, and the probe comprises one selected from the group consisting of nucleic acids, peptides, saccharides, lipids, cells, and fragments of these materials, and wherein the probe is immobilized on a circumferential side wall of the substrate. Examples of the artificial biologic material include synthetic DNAs, synthetic RNAs, synthetic peptides, synthetic polynucleotides, synthetic sugar chains, and functional materials containing complexes of these materials. The biologic material herein is preferably derived from an object substance selected from the group consisting of microorganisms, ligands, nucleotides, antibodies, antigens, proteins, peptides, carbohydrates, polysaccharides, receptors, drug targets, vegetable or animal cells, organelles, bacteria, pathogenic organisms, antibiotics, drugs, toxins, naturally-occurring substances, test compounds, and fragments of these substances. The probe is immobilized on the substrate preferably by printing, drawing, spraying, affixation, wrapping, adsorption, a chemical reaction, or synthesis in situ. The resulting biological chip may be cut vertically to its central axis to thereby prepare plural second biological chips.

The biological chip preferably further comprises an index mark indicating the position of the probe on the surface of the substrate.

In a preferred embodiment, the index mark comprises plural index marks, the probe comprises plural probes immobilized at plural positions in the substrate, the plural positions of the immobilized probes are separated into plural groups, and the index marks are so arranged at predetermined positions on the substrate as to distinguish one group from another.

The probe preferably comprises plural probes and includes at least one internal standard probe. The “internal standard probe” herein is a probe that reacts with not a sample material but only an internal standard material added in the medium containing the sample to be reacted with the biological chip.

The present invention further provides, in a second aspect, a biological chip assemblage comprising stacked plural plies of the biological chip, in which the biological chips are so stacked that the central axes of the columns or cylinders substantially coincide with each other.

A spacer is preferably arranged between adjacent two biological chips.

In addition, the present invention provides, in a third aspect, an incubator serving to bring a sample into contact with the biological chip or the biological chip assemblage, the sample being reactive with at least one of the probes, to thereby carry out a reaction between the sample and the at least one of the probes, in which the incubator is so configured as to keep the central axis of the column(s) or cylinder(s) of the biological chip or biological chip assemblage being substantially horizontal and to rotate the biological chip or biological chip assemblage around the central axis, and the incubator is so configured as to immerse a vertically lower part of the biological chip or biological chip assemblage in a medium containing the sample.

The present invention further provides, as a first assay device, an assay device for determining at least one of physical properties, chemical properties, and biological properties of a molecule bound to the probe on the biological chip or the biological chip assemblage, in which the assay device comprises a measuring section and is so configured as to rotate the measuring section along the circumferential side wall of the column(s) or cylinder(s) around the central axis of the column(s) or cylinder(s). The physical properties herein include, for example, emission intensity, absorbance, and electric conductivity. The chemical properties include reactivity with a specific reagent. The biological properties include the types of products produced by the action of a predetermined enzyme.

The present invention also provides, as a second assay device, an assay device for determining at least one of physical properties, chemical properties, and biological properties of a molecule bound to the probe on the biological chip or the biological chip assemblage, in which the assay device is so configured as to carry out an assay while rotating the biological chip or the biological chip assemblage around the central axis of the column(s) or cylinder(s).

The assay devices are preferably so configured as to evaluate measured results in consideration of biological meanings of the respective probes and to display the evaluation results. The assay devices are also preferably so configured as to carry out assays on plural probes of plural biological chips simultaneously, when they are used for the assay of the biological chip assemblage.

The assay devices preferably further comprise a mediator between the measuring section and the biological chip or biological chip assemblage. The mediator can be, for example, a membrane, a plate or sheet, a rod, or a powder, of which a membrane is typically preferred. The membrane for use herein preferably comprises a photo-sensitive material, a photostimulatable luminophor, a composite of a luminophor and a photosensitive material, or a filter.

The present invention further provides, as a second biological chip, a biological chip comprising a columnar or cylindrical substrate; and plural containers arranged on the circumference of the substrate. The containers are preferably incubation cells. The biological chip is preferably so configured as to carry out a reaction in the container and to determine the absorbance of a reaction material in the container according to at least one of microphotometry and a confocal system. In this case, the containers are preferably translucent, because the assay can be conducted from outside of the containers.

The containers are preferably detachably arranged on the substrate.

The containers preferably contain assay reagents.

In a preferred embodiment, the assay reagents are solid.

The assay reagents are each preferably a reagent which specifically reacts with a material to thereby determine the presence or absence of the material and to determine one of the quantity and activity of the material, wherein the material is selected from the group consisting of molecules and genes including or encoding GOT, GPT, LDH, CPK, ALP, gamma-glutamyl transpeptidase, LAP, BUN, CRE, Ch-E, Na, K, Cl, Ca, P, Fe, Mg, ammonia, sialic acid, blood sugar (BS), HbA1C (hemoglobin A(1C)), amylase, bilirubin, total protein, albumin, uric acid, cholesterol, neutral lipid, HDL cholesterol, LDL, CRP, digoxin, theophylline, valproic acid, phenytoin, thyroid hormone, TSH, HBs (hepatitis B surface) antigen, HCV (hepatitis C virus) antibody, GH, LH, FSH, prolactin, ADH, ACTH, renin activity, aldosterone, myoglobin, ANP, BNP, erythropoietin, insulin, PSA, CEA, CA 19-9 (carbohydrate antigen), and AFP, respectively. The reagents can also be reagents that specifically react with any of materials serving as index for finding human diseases or materials serving as index for determining whether the health of a human is good or bad, in addition to the materials included in the group, to thereby determine the presence or absence of the material and to determine one of the quantity and activity of the material.

At least one of the containers preferably contains plural assay reagents of different types.

The containers preferably have identifying marks on their outer surfaces for distinguishing one container from another. For example, it is useful to catalog the patient identification information, the type of material to be measured, and assay date into the identifying marks.

As is described above, the biological chips according to the present invention use the circumferential side wall of a column or cylinder as probes or reactors. Accordingly, a variety of probes or reactors can be easily prepared on one chip in a short time, and reactions with a sample and assays after the reactions can be easily carried out in a short time.

### BRIEF DESCRIPTION OF THE DRAWINGS

FIG. 1 is a schematic perspective view of a discoidal biological chip according to First Embodiment.

FIG. 2 is a schematic perspective view of a multilayer discoidal biological chip according to Second Embodiment.

FIG. 3 is a schematic perspective view of a biological chip assemblage according to Third Embodiment.

FIG. 4 is a schematic view of an incubator for the biological chip assemblage according to Second Embodiment.

FIG. 5(a) is a schematic view of a biological chip according to Fourth Embodiment, and FIG. 5(b) is a schematic view of the queue of incubation cells.

FIG. 6 is a schematic view of the principal part of a DNA chip reader according to Second Embodiment.

FIG. 7 is a schematic view of the principal part of another DNA chip reader according to Second Embodiment.

### BEST MODE FOR CARRYING OUT THE INVENTION

Initially, the features of the present invention will be schematically illustrated with reference to some embodiments.

According to an embodiment, a biological chip comprising a discoidal or cylindrical substrate and probes arranged and immobilized on a circumferential side wall of the substrate is prepared, which probes each comprise a nucleic acid, a peptide, a saccharide, a lipid, a chemical substance, or a fragment thereof. The disc or cylinder is rotated so as to attain rapid intake of massive assay information obtained from plural probe groups arranged on the circumferential side wall. Mass-production of a lot of chips or large-capacity chips is accomplished by constructing the disc or cylinder from a large number of thin sheets or by stacking plural thin sheets into a disc or cylinder. According to another embodiment, system simplification and miniaturization, and resource saving are realized in blood tests by using a biological chip having incubation cells arranged thereon.

Specifically, probes are continuously immobilized linearly in parallel with the central axis of the disc or cylinder on the circumferential side wall of the discoidal or cylindrical structure, and the disc or cylinder is then rotated around the central axis by a predetermined angle. Next, probes of another group are continuously immobilized linearly in parallel with the central axis on the circumferential side wall. Probes of plural types can be immobilized over a wide region in one step by repeating this procedure until the disc or cylinder is rotated 360 degrees.

A lot of chips having the same configuration can be easily prepared by constructing the cylinder or disc from an assemblage of stacked thin sheets and disassembling the assemblage after immobilizing the probes; or by cutting the cylinder or column into thin layers after immobilizing the probes.

Probes can be immobilized by photolithography in a similar manner. Specifically, for a nucleic base of a single kind, light is applied in parallel with the central axis of the disc or cylinder to thereby remove the photo-reactive protecting group, and the disc or cylinder is rotated around the central axis by a predetermined angle or a predetermined distance. Then, light irradiation and rotation of the disc or cylinder are conducted again. The series of procedures is repeated until the disc or cylinder is rotated 360 degrees, and the specific base is allowed to bind with the sites from which the photo-reactive protecting group has been removed. In this case, a single linear photomask is all that is required to irradiate light.

Drawing a pattern of bases can be carried out by placing four units alternately on the line, which four units are used for continuous drawing and comprise four bases, i.e., adenine, guanine, cytosine, and thymine, respectively. These four bases are each protected by a photo-reactive protecting groups and are dispersed in a medium. The production process can be carried out on a plane in a similar manner as follows. Rolled strip or filamentous substrates are arranged on a plane, and probes of one kind are immobilized in a direction perpendicular to the longitudinal direction of the strip or filamentous substrates. After the probes are immobilized up to one end of the substrates, the substrates are shifted by a predetermined distance, and probes of another kind are immobilized in a direction perpendicular to the longitudinal direction of the strip or filamentous substrates. This procedure is repeated. Finally, constitutional strips or filaments are separated. Thus, a multiplicity of chip constitutional parts can be prepared in one step. Namely, an array of probes of plural kinds can be prepared on a plane or a cylinder. When probes are formed on a circumferential surface of a disc or cylinder, the disc or cylinder is rotated preferably while moving plural probe-immobilizers at specific intervals in parallel with the central axis of the disc or cylinder. This can further increase the working efficiency.

In another preferred embodiment, discs or cylinders of different types having immobilized different probes are stacked to constitute a large-capacity biological chip. The discs or cylinders preferably have some labeling for identifying the arrangement and order of probe-immobilized sites. The discs or cylinders also preferably have engagement comprising some notches or convex and concave portions for correcting the distortion or misregistration of stacked layers (discs or cylinders). A spacer of a material different from that of the discs or cylinders, or a coating typically by plating or sputtering is preferably arranged between layers for easier separation, identification, or isolation of the respective layers.

The biological chip having probes regularly and precisely arranged lengthwise and crosswise on the circumferential side wall of the disc or cylinder is allowed to react with a medium (fluid) containing the sample in a variety of reaction processes. By utilizing the rotation of the disc or cylinder, the assay using this biological chip can be carried out with a less amount of the sample or the fluid more stably at higher sensitivity than the case where incubation is conducted two-dimensionally on a plane as in conventional DNA chips.

In addition, the biological chip can be configured into an assay system with an assay device arranged around the disc or cylinder. The assay system is so configured as to rotate the disc or cylinder or to move the assay device around the disc or cylinder to thereby analyze the results of biological or physicochemical reactions between the sample and probes arranged on the disc or cylinder. Thus, the assay system can be constructed more easily, more simply, and more sophisticatedly than conventional equivalents. The results of biological or physicochemical reactions between the sample and probes can be determined using a detection chip. The detection chip is preferably in the form of a cylinder or disc and preferably has one or more projections on its circumferential side wall. The detection chip is preferably arranged in the vicinity of the biological chip so that the one or more projections face the probes on the circumferential side wall of the cylindrical or discoidal biological chip. When the biological chip carries probes arranged along the central axis direction of the chip, the detection chip preferably has projections in a number corresponding to that of the probes, and the biological chip and the detection ship are preferably so arranged that the probes and the productions face each other in a one-to-one manner. More preferably, the biological chip and the detection chip are constructed into a measuring/analyzing device or system which further comprises a filter membrane between the biological chip and the detection chip. The measuring/analyzing device or system is so configured as to analyze the results by an optical, electrical, chemical, physical, or biological process or device. The measuring/analyzing device or system has much higher precision and higher sensitivity and can be more easily operated than conventional equivalents in which two-dimensional biological chips are measured and analyzed.

Thus, a simple assay system can at least be constructed. The assay system is so configured as to assay the results of reactions between the probes and the sample only by the action of rotating the disc or cylinder around its axis or moving the disc or cylinder along its central axis. In addition, the assay can be continuously carried out even on plural samples. Specifically, the data can be acquired at high speed according to a line scanning system by stacking plural discoidal biological chips into a multilayer chip assemblage and by aligning probes in the respective layers of the assemblage in straight lines. Moreover, scanning of overall the probes can be easily conducted at high speed according to the system using the circumferential side wall of the disc or cylinder only by the combination of rotation and linear movement of the chip. This enables rapid carrying out of pre-scanning before the binding of the sample with probes, assay scanning upon binding of the sample, and post-scanning after the sample is removed. Thus, the results in more precise accordance with the true or actual binding of the sample to probes can be obtained.

The acquired and analyzed results of reactions between the probes and the sample cannot substantially exhibit their significances, if they are simply present as isolated information. Namely, the reaction results can become very important information to comprehend biological phenomena and carry out various interpositions, only after the results are subjected to an analyzer and software that compare the results with the inherent properties of the groups of probes and thereby determine biological meanings of the results and/or subjected to comparison of the results between plural samples. Specifically, the total value of the assay system can further be increased by feeding the reaction results to an excellent information analysis system. When plural samples are assayed, plural assays are preferably carried out concurrently. This is useful for time or resource savings and for precise relative evaluations.

Of tests using biological chips, blood biochemical tests particularly require precise determination of the quantity and/or concentration in evaluation of reaction processes or reaction results, as in enzymatic activities. In these tests, the assay system must be so configured not only to dip the chip in a medium (fluid) relating to the reaction but also to precisely set the total amount and material concentration of the medium. In particular, only a trace error results in a large variation of the measured value in such a small-scale reaction system in the chip. Accordingly, incubation cells having uniform dimensions and uniform and small capacities are preferably arranged regularly on the circumference of the chip. This enables precise and rapid reactions and precise and rapid assays of the reaction results using a very small amount of the sample in a very small-sized system, as compared with the case where reactions and assays are carried out using a conventional automatic sampler to inject a sample or a reaction mixture into the reactor.

For constituting a precise and simple reaction assay system, it is useful to place reaction fluids in the incubation cells in advance using a dedicated production device. For one-step reactions, predetermined small, precise quantities of reaction fluids are placed in the incubation cells, respectively.

For two-step reactions, the reactions can be carried out in the following manner. Dual-chamber incubation cells each having a partition wall are prepared; a first reaction is conducted in one chamber; and then a second reaction is conducted after breaking the partition wall. Alternatively, an easily breakable container containing a reagent for the second reaction is prepared and is broken after the first reaction. Further alternatively, the reagent for the second reaction is placed in an incubation cell typically according to an ink-jet system or a micro-pipette system. Small amounts of fluids in the incubation cells can be effectively admixed by applying vibration generated by a vibrator or an acoustic wave or by centrifugal rotation. When the assay is conducted by absorptiometry in a chip system including incubation cells, the assay can be carried, for example, by measuring light through walls of incubation cells made from a translucent material, by sampling the reaction mixture using a mini-sampler and measuring the absorbance thereof typically in a cuvette, or by inserting a microprobe for measuring the absorbance into the reaction mixture in incubation cell. The confocal method is also preferred as the measuring process, because the assay system can be reliably located in the reaction mixture.

The reaction between a sample and, for example, a reaction reagent must be conducted in a liquid phase in the biological chip. However, a liquid reagent, if used, may evaporate during storage or may decrease in its activity with elapse of time. To avoid these problems, the incubation cells preferably contain necessary reagents in the form of solids which have been prepared typically by freeze-drying. According to this configuration, reactions can be proceeded using reagents having precise concentrations and precise activities only by adding the sample or a diluted fluid thereof to the incubation cells. Two-step reactions can be conducted according to this technique, for example, by separating an incubation cell with a partition wall and breaking the partition wall upon initiation of the second reaction; by arranging a reservoir for the second fluid, which reservoir can be broken typically by osmotic pressure; or by placing the second reagent into the incubation cell using an ink-jet system or a micro-pipette system.

For carrying out an enzyme-linked immunosorbent assay (ELISA), a chip carrying an immobilized first antibody is incubated with the sample, is rinsed, and is incubated with an enzymatically labeled second antibody, is rinsed, and a photochemical reaction is then measured.

In medical or biological assays, items to be analyzed are often selected from a lot of items. In this case, it is preferred that incubation cells are so configured as to be detachable and to carry out plural different reactions, and items to be analyzed are selected by detaching or attaching the detachable incubation cells in a certain combination. This avoids unnecessary consumption of the supply and eliminate waste in assay, as compared with the case where items are selected indiscreetly. For the sake of convenience, items inherent to, for example, electrolytes, liver functions, renal functions, and myocardiopathy are preferably categorized as groups, incubation cells corresponding to these items are also preferably categorized as groups. Thus, total items to be analyzed can be selected as a combination of some of these groups.

When the biological chip carries detachable incubation cells, the respective incubation cells must have the function of displaying and identifying themselves so as to indicate which cell is for measuring what item of which patient. Accordingly, the incubation cells are preferably labeled to indicate the origin of the sample, items to be analyzed, and test date. This enables concurrent assays of different items on different subjects. The labeling of the incubation cells can be achieved, for example, by applying labels to the incubation cells or directly printing data on the incubation cells.

When the biological chip comprises plural probes on the circumferential side wall of the disc or cylinder, the chip is preferably configured in the following manner; the probes are immobilized at positions on the circumferential side wall, which positions are separated into groups at identical intervals along the rotational direction of the disc or cylinder; the respective groups have different marks for identifying the types of the probes varying from one group to another; and index marks for showing the positions where probes are to be immobilized are arranged along the rotational direction of the disc or cylinder. The position of probes of the target type to be assayed can be recognized beforehand by reading and identifying the index marks. Thus, the assay precision can be increased, and the accuracy and speed in continuous assay can be dramatically improved.

When the biological chip is a multilayer assemblage comprising stacked cylinders or discs, indicators for recognizing interlayers are preferably applied, or a mediator of a material different from that of the discs or cylinders is preferably arranged for sectioning layers. In this case, probes are immobilized at positions of groups arranged along the indicators. Thus, the probes can be regularly and precisely arranged and immobilized lengthwise and crosswise on the circumferential side wall.

In a preferred embodiment, a biological chip comprises an assemblage of stacked cylinders or discs having a reflective surface; marks (index marks) for indicating the positions at which groups of probes are to be immobilized; interlayer indicators (tracking marks); an optically transparent member arranged on the circumferential side wall; and plural measuring sections comprising DNA probes immobilized on the optically transparent member, in which the measuring sections are arrayed along the interlayer indicators and the index marks and are sectioned along the array direction, the index marks varying from one section to another are arranged in the respective sections, and the index marks are readably recorded. This biological chip enables more easy and precise optical scanning.

As yet another embodiment, a biological chip reader is so configured as to scan probes immobilized on the circumferential side wall of a cylinder or disc structure of a biological chip by rotating the biological chip around its central axis. In a preferred embodiment, the biological chip reader is so configured as to scan probes immobilized on the circumferential side wall of a cylinder or disc structure of a biological chip by rotating the biological chip around its central axis; has the function of tracking servo for following a tracking mark upon scanning of the probe-immobilized sites; and is so configured as to recognize the probe-immobilized sites independently in accordance with the index marks upon scanning of the probe-immobilized sites. The biological chip reader more preferably further comprises a focusing servo mechanism that can follow the change in distance between the read head and the disc or cylinder of the biological chip with the rotation of the biological chip.

As still another embodiment, a biological chip reader comprises a rotation mechanism for holding a biological chip and rotating the chip around its central axis, the biological chip comprising a cylindrical or discoidal structure, and probes immobilized on the cylindrical or discoidal structure; a traveling mechanism for changing the position of the chip in parallel with the rotation axis; a light source; an irradiation optical system for applying light emitted from the light source to the biological chip; a translation device for changing the relative position between the light source and the biological chip; an optical system for detecting reflected light from the biological chip; an optical system for detecting fluorescence from the probe-immobilized sites of the biological chip; and controller for controlling these devices, mechanisms, optical systems, and other components, in which the optical system for detecting reflected light has the function of tracking servo, whose position is controlled by the controller based on the marks for indicating the probe-immobilized sites (tracking marks) arranged on the biological chip; and the function of reading indexes by the action of the controller based on the index marks arranged on the biological chip, the fluorescence detection optical system comprises a photo-detector and an aperture for allowing fluorescence to selectively transmit to the photo-detector, and the reader is so configured that signals from the photo-detector enter the controller, and information from a specific probe-immobilized site is independently read out according to the index mark upon scanning of the probe-immobilized sites. As another embodiment, the optical system for detecting reflected light can further comprise a focusing servo function for following the change in surface shape of the biological chip with the rotation of the biological chip.

The biological chip reader can further comprise a read drum facing the discoidal or cylindrical chip and can be so configured as to rotate the drum with the chip to thereby continuously assaying and obtaining data from a specific limited area at the contact face between the drum and the chip. This reader can perform assays with higher sensitivity of the information obtained from the probes, higher precision, and reduced background.

Specifically, the noise can be reduced and the background can be reliably set, because the contact between the chip and the read drum is limited to a tangent line. In this configuration, a filter membrane structure can be arranged at the contact face between the chip and the read drum. Alternatively, the filter can be so configured as to serve as a measuring medium. Preferred examples of materials for the chip, filter, and drum facing the chip are photosensitive media, photostimulatable luminophors, electrically conductive components, light-emitting substrates, adhesive materials, and insulative materials which comprise, for example, carbon nanotubes, organic light-emitting materials, metallic powders, and membranes and exhibit properties featuring the materials. By using these materials, measured information from the probe-immobilized sites can be collected easily with high sensitivity by the action of, for example, an optical, electrical, physical, chemical, or biological detection device or process.

The biological chip is preferably kept in contact with the read drum or the interposing membrane. By this configuration, information can be sequentially collected from the circumferential side wall of the chip along the circumferential direction by rotating or scrolling one of the two members. A flexible structure is preferably used in combination so as to prevent slipping between the two members.

The present invention will be illustrated in further detail with reference to several embodiments below, which by no means limit the scope of the present invention.

### FIRST EMBODIMENT

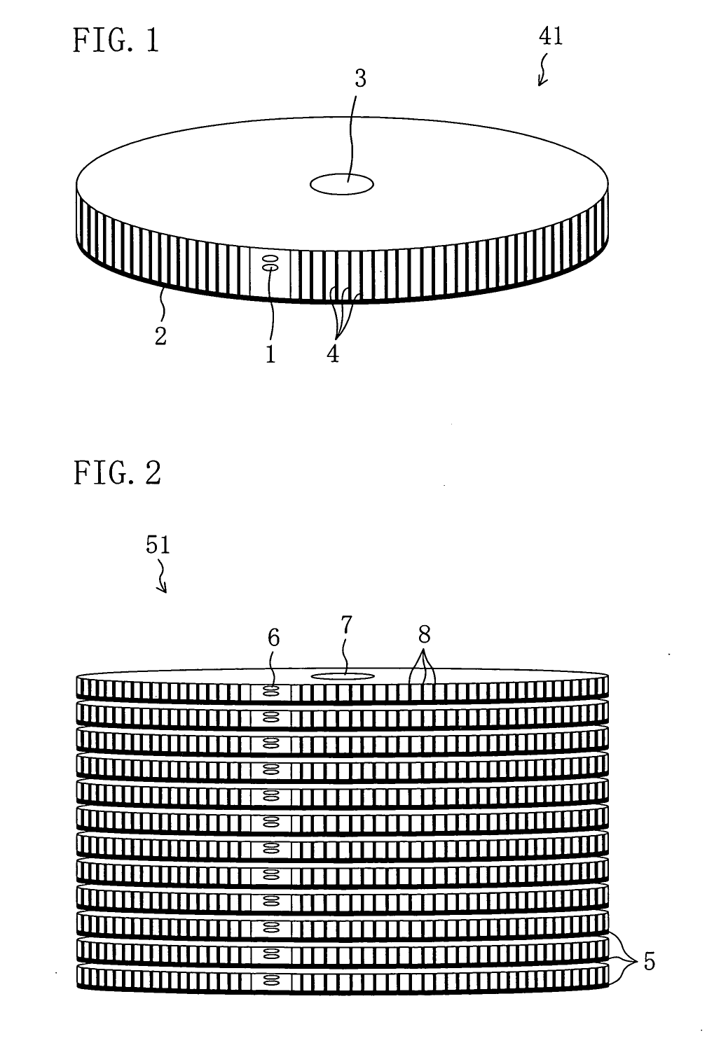

FIG. 1 is a schematic view of a biological chip 41 as First Embodiment of the present invention.

The biological chip 41 is in the form of a disc having a diameter of 2 cm and has a hole 3 at its center part. The hole 3 serves to fix the biological chip 41 to a spindle for rotation. The biological chip 41 according to First Embodiment is a DNA chip.

The disc serving as a substrate is made from a polycarbonate and has a thickness of about 5 mm. The biological chip 41 can also be referred to as a column although it is thin. It can also be referred to as a cylinder, because it has the hole 3 at its center part. The circumferential side wall of the polycarbonate disc carries dimples 1 as an index mark at its upper part, and a tracking mark 2 formed by coating at its lower part. Probes 4 are arranged and immobilized linearly on the circumferential side wall in parallel with the central axis of the disc. The plural dimples 1 as an index mark are arranged on the circumferential side wall and have different dimensions such as shapes and/or depth so as to distinguish dimples of different groups that are arranged separately at some intervals in the circumferential direction of the disc. Some index marks are hidden and not shown in FIG. 1. The “index marks” as used herein serve to indicate the arrangements and positions of the groups of probes 4 arranged on the chip. When the chip comprises plural probes 4 as in this embodiment, the probes 4 are separated into plural groups, and index marks are arranged between adjacent groups of probes 4 so as to distinguish one group from another by the index marks. Specifically, the plural index marks are so arranged that they are distinguishable from each other, and the groups of the probes 4 corresponding to these index marks are also distinguishable. The tracking mark 2 serves as a registration mark for determining proper positions of the probes upon read out of signals, as mentioned below.

An aluminum reflective layer 500 nm thick prepared by vapor deposition is arranged on the surface of the circumferential side wall of the disc. This makes the surface of the circumferential side wall of the disc reflective. The tracking mark 2 has a width of 20 μm, and the dimples 1 have a depth about one-quarter of the wavelength of a light source used for reading the index. A glass layer as an optically transparent member is arranged to a thickness of 50 μm on the aluminum reflective layer. The glass layer in the DNA chip 41 according to this embodiment is formed from a borosilicate glass by sputtering.

Multiple probes (DNA probes) 4 are arranged on the surface of the glass layer, and adjacent probes 4 are arranged at regular intervals, in other regions than the dimples 1.

As is described above, the side wall are separated into plural sections by the plural index marks 1, and one section comprises an index mark 1 and ninety-six probe-immobilized sites. Thus, the probe-immobilized sites are separated into sections in a row direction of the arrangement, and each section has a specific index mark.

In a preferred embodiment, a blank probe is arranged in one of the probe-immobilized sites; a specific artificial probe as internal standard is arranged in another; and a predetermined amount of a synthetic nucleotide is added to the sample before assay, which synthetic nucleotide is reactive only with the internal standard probe in the after-mentioned step. By satisfying this, measured values can be guaranteed, because the measured values of the blank and of the internal standard can be set at zero and the specific standard value, respectively, upon assay through a hybridization with the medium containing the sample. The “blank” as used herein is a material that is unreactive with the sample such as a DNA and includes, for example, a buffer solution such as a salmon sperm DNA solution.

Next, the method for preparing the DNA chip 41 as the biological chip by photolithography will be illustrated in detail.

Initially, light emitted from a tracking/index light source for position detection passes through a collimator lens and a semi-transparent mirror (half mirror), is reflected by another semi-transparent mirror, is collected by a lens, and is applied to the aluminum reflective surface of the DNA chip 41. The collimator lens, the two semi-transparent mirrors, and the lens constitute an irradiation optical system for applying light emitted from the position-detection light source to the circumferential side wall of the disc.

The reflected light from the aluminum reflective surface passes through a lens and a semi-transparent mirror and is introduced into the tracking/index detection optical system by the action of another semi-transparent mirror. The tracking/index detection optical system serves to read the tracking mark and carry out tracking servo in the same manner as the DNA chip reader. An index mark is then read out, and the irradiation system applies light to the DNA chip 41 based on the position of the index mark, and the position of the applied light on the DNA chip is outputted.

The controller controls On/Off of the light source for removing photo-reactive protecting group based on the position information fed from the tracking/index detection optical system. The optical system of the DNA chip production unit comprises integrated components including a light source, a photoreceptor, and an optical element and is so configured as to apply light to any arbitrary position on the circumferential side wall of the DNA chip 41. Specifically, the DNA chip production unit comprises a light source for applying light to the circumferential side wall of the DNA chip 41; and a drive for changing the relative position between the light source and the DNA chip 41.

Amino groups are introduced overall the surface of the glass layer of the DNA chip 41 using a silane coupling agent. The amino groups each have a bonded photo-reactive protecting group having the same function as those used in conventional techniques for synthesizing DNA probes by photolithography. The DNA chip 41 is fixed to a spindle and is controlled in movement so as to carry out a linear monoaxial movement and a rotational movement.

The coordinates on the circumferential side wall of the DNA chip 41 are detected using the tracking/index detection optical system, and the light source for removing photo-reactive protecting group is turned on at the time when a region to which a desired nucleotide is to be bound is located directly bellow the region where light is to be applied. The light emitted from the light source passes through a lens and is applied to the desired region on the DNA chip 41 so as to remove the photo-reactive protecting group in the region. By moving the DNA chip 41 in parallel with the central axis, a series of regions where the photo-reactive protecting group has been removed is formed linearly on the circumferential side wall. This procedure is repeated on each region to which a specific base is to be introduced. After finishing the treatment of the entire circumferential side wall of the disc, a solution containing the nucleotide to be bound is charged in the unit.

As a result, the predetermined nucleotide is bound only to the group of linear regions from which the photo-reactive protecting group has been removed.

The above-mentioned step is carried out once on every nucleotide (base), i.e., A, G, T, and C. By repeating the series of procedures until a desired nucleotide sequence is obtained, DNA probes 4 can be immobilized all the target regions of the DNA chip 41.

This immobilization is generally carried out in the following manner. Initially, the photo-reactive protecting group is removed in a predetermined region while rotating the DNA chip little by little. After the entire circumferential side wall is treated, the desired nucleotide is bounded. This is repeated on every base, i.e., A, G, T, and C as a first base of each probe of the programmed plural nucleotide probe array. Specifically, one base is elongated overall the circumferential side wall by repeating the process of light irradiation and nucleotide bonding a total of four times.

In this embodiment, the probe oligonucleotides are synthesized and immobilized on the surface of the circumferential side wall of the DNA chip 41 using a photoreaction.

However, the probes can also be immobilized using an ink-jet device or an imaging mechanism that have a known positional relation with the regions to be irradiated with tracking light.

The production unit according to this embodiment preferably further comprises a focusing servo mechanism that serves to keep the distance between the lens and the circumferential side wall of the DNA chip 41 even when the surface level of the circumferential side wall of the DNA chip varies with the rotation. This enables the immobilization of the DNA probes 4 even when the surface of the DNA chip 41 is uneven and the position (level) of the surface of the DNA chip 41 varies with the rotation. The distance between the surface of the DNA chip 41 and the lens can be determined, for example, by an astigmatic method, a Foucault process, a knife edge method, or an SSD method.

The DNA chip 41 according to this embodiment has a substrate in the form of a disc, but the substrate can also be, for example, in the form of a cylinder or tube which is a disc having a hole at its center part. Likewise, the index mark dimples 1 and the tracking mark 2 in this embodiment are formed by compression molding and coating, respectively, but they can be formed, for example, by injection molding and two-piece process. The aluminum reflective layer is deposited on the circumferential side wall of the disc by vapor deposition in this embodiment, but it can also be deposited, for example, by sputtering.

The material for the disc can be, for example, a resin such as a poly(vinyl chloride) or an acrylic resin; glass; a metal such as aluminum; silicon (Si); or a porous ceramic. The disc is preferably formed from a material having a high reflectivity, such as aluminum or silicon, because this eliminates the need of deposing a reflective film on the surface of the disc.

In this case, the index mark dimples 1 and the tracking mark 2 are formed typically by compression molding, etching, or cutting. The optically transparent borosilicate glass is formed on the DNA chip 41 according to this embodiment by sputtering, but it can also be deposited typically by vacuum deposition or chemical vapor deposition (CVD).

The optically transparent member used in this embodiment is the borosilicate glass arranged on the reflective surface of the circumferential side wall of the disc in the DNA chip 41, but the optically transparent member can also be, for example, another glass such as quartz glass or Pyrex (trademark) glass; or an optically transparent resin such as a polycarbonate, a poly(vinyl chloride), or an acrylic resin. When the substrate is made from silicon, the optically transparent member can also be formed by thermally oxidizing the silicon substrate to form a SiO₂ film as the optically transparent member after forming the tracking mark and index marks on the surface of the silicon substrate typically by etching. In addition, the efficiencies of DNA immobilization and hybridization can be improved by using a porous material, and the chip can be so configured as to provide for electrical or physical assay by treating the surface with carbon nanotube or gold thin film. The number of probes to be immobilized in one section can be another number than ninety-six.

### SECOND EMBODIMENT

According to Second Embodiment, plural plies of a thin disc or cylinder 1 mm thick are stacked to thereby prepare plural chips having an identical configuration. FIG. 2 is a schematic view of the chips. An aluminum reflective layer 500 nm thick as a reflective film is arranged by vapor deposition on the circumferential side wall of each disc having a thickness of 1 mm and a diameter of 2 cm, as in First Embodiment. This makes the surface of the circumferential side wall of the discs reflective. Each disc has a hole 7 as in First Embodiment. A tracking mark 5 has a width of 20 μm, dimples 6 as index marks have a depth about one-quarter of the wavelength of a light source used for reading the index. A glass layer as an optically transparent member is arranged to a thickness of 50 μm on the aluminum reflective layer. Hundred plies of the discs having this configuration are sequentially so stacked that the respective index marks are aligned on a line in parallel with the central axes of the discs and the respective central axes substantially coincide with each other.

After the completion of the operation, one of the group of probes 8, 8 is continuously linearly immobilized on the circumferential side wall in parallel with the central axis of the biological chip 51 by printing, in which the arrangement and sequence of the group of probes have been determined corresponding to the respective index marks. Next, the discs are rotated anticlockwise by 0.6 degree, and another probe 8 is continuously immobilized by printing using an ink-jet printer. The continuous immobilization of probes and the disc rotation are repeated alternately 600 times. Thus, the probes 8, 8 are arranged in the circumferential direction of the disc at intervals of about 105 μm. Assuming that the printed image has a width of 20 μm, each probe 8 is immobilized on the discs with an area of 0.02 mm² per one chip. The discs after immobilization of the probes are sequentially taken out from the central axis and thereby yield hundred DNA chips 51, 51 of the same lot having the same configuration, each of which carries probes of six-hundred types immobilized all around the circumferential side wall. Spacers are arranged between the respective DNA chips 51, 51 so as to enable the DNA chips 51, 51 to be taken out easily, while the spacers are behind the DNA chips 51, 51 and are not shown in the figure.

Next, a chamber for carrying out the hybridization between the DNA chips 51 according to this embodiment and a sample will be illustrated. A discoidal chip assemblage 11 has a thickness of 21.9 mm and comprises twenty plies of the DNA chips 51, 51 having a diameter of 2 cm according to this embodiment. The discoidal chip assemblage 11 is so tilted that the central axis is horizontally oriented, and the assemblage is arranged in a chamber 13 having a curved bottom, as illustrated in FIG. 4. A shaft 12 is placed in a through-hole arranged in the vicinity of the central axis of the discoidal chip assemblage 11. The shaft 12 supports the discoidal chip assemblage 11 so that a small clearance is formed between the circumferential side wall of the discoidal chip assemblage 11 and the chamber. An aliquot of a medium containing a sample 14 (200 μl) is injected into the clearance. A stable and reliable hybridization can be achieved between the probes 8, 8 and the sample by carrying out incubation while rotating the discoidal chip assemblage 11 around the shaft 12. At the time when sufficient binding between the sample and the probes 8, 8 is achieved, the discoidal chip assemblage 11 is taken out from the chamber 13, is rinsed, and the amount of the bound sample is determined on each of the DNA chips 51, 51. More specifically, the discoidal DNA chips 51, 51 carrying the immobilized oligonucleotide probes 8, 8 are incubated with a fluid (sample) containing a DNA marked with a fluorescent dye. The DNA chips 51, 51 are then rinsed while being rotated. By carrying out reactions with the sample in this manner, all the probes 8, 8 can be exposed to the sample under the same conditions. This enables reactions of the respective probes 8, 8 under equivalent conditions and significantly increases the opportunity of contact between the probes 8, 8 and sequences in the sample to be bound. The humidity in the incubator (chamber) is preferably adjusted so as to prevent drying of the chips during operation.

Next, a system for analyzing the reactions of the DNA chips 51, 51 will be illustrated. Initially, the discoidal chip assemblage 11 comprising the stacked DNA chips 51, 51 is attached to a DNA chip reader. The DNA chip reader comprises a mechanism for holding the discoidal chip assemblage 11 and rotating the same around the disc axis by the action of a spindle and a servo motor; and a mechanism for scanning probe-immobilized sites distributed over plural layers by the action of monoaxial movement.

The DNA chip reader has an optical system comprising integral components including a light source, a photoreceptor, and an optical element. The integrated optical system is so configured as to move in parallel with the central axis of the discs at one position of the circumferential side wall of the DNA chip 51. Specifically, the DNA chip reader comprises a drive for moving the position of the light to be applied to an arbitrary position of the circumferential side wall of the DNA chip 51. The light emitted from the light source passes through a collimator lens and a semi-transparent mirror, is reflected by a mirror, is collected by a lens and is applied to the aluminum reflective surface of the DNA chip 51. The reflected light from the aluminum surface passes through a lens and a mirror and is introduced into a tracking/index detection optical system by a semi-transparent mirror.

The tracking/index detection optical system precisely detects the positions of probes on the circumferential side wall of the chip by any of, for example, a push-pull method, a wobble method, or a three-beam method used in regular tracking servo mechanisms and carries out tracking servo so that the light applied to the aluminum reflective surface of the DNA chip 51 follows the tracking mark 5. Consequently, the irradiated light does not deviate from the probe-immobilized site even when the central axis of the DNA chip 51 deviates from the center of the DNA chip 51 or the circumferential side wall of the DNA chip 51 has an uneven surface. This can be achieved by controlling the position of the integrated optical system based on signals from the tracking/index detection optical system.

The tracking/index detection optical system also serves to read the index marks 6 and output signals indicating the position coordinates on the DNA chip 51. As is described above, the index marks 6 have a depth one-quarter of the wavelength of the light source. The optical path difference between light emitted from the light source and reflected at the upper part of the index marks 6 and light reflected at the bottom thereof is half the wavelength of the light source. The total reflected light from the index marks 6 has a smaller intensity than that of light applied to other regions than the edges of the index marks 6. The index marks 6 differ from each other from one section to another as described above, and the change in intensity of the reflected light per each index mark 6 can be used as a signal for indicating the position coordinate. Namely, the index marks 6 having a step (depth) of one-quarter of the wavelength of the light source are thus readably recorded.

When the DNA chip 51 moves, the light reflected by the mirror is applied to a region at which a DNA probe 8 is immobilized on the surface of the circumferential side wall of the DNA chip 51, and hybridization occurs in the region, fluorescence is generated on the probe-immobilized site of the DNA chip 51 by the action of the applied light. The resulting fluorescence is collected by a lens, is reflected by a mirror and is focused to an aperture by a lens. The size and position of the aperture are set so as to selectively transmit the fluorescence from the surface of the DNA chip 51. The aperture has the same configuration as in regular detection methods using confocal microscopes. Accordingly, noise light such as light from the aluminum reflective surface, contaminated light, and flare from intermediate optical systems can be efficiently blocked by the action of the aperture. The fluorescence passed through the aperture is detected by a photo-detector. When the detection of fluorescence is accomplished at the uppermost region to be measured in a specific region on the circumferential side wall, the discoidal chip assemblage 11 comprising a series of the DNA chips 51, 51 is moved upward, and registration and assay are sequentially carried out. Thus, the last 20th probe 8 is registered and assayed.

The reader may further comprise an arithmetic unit. The arithmetic unit serves to output the position of the probe-immobilized site and the probe number in correspondence with the fluorescence intensity as a result of hybridization, based on the position information from the tracking/index detection optical system and the output from the photo-detector.

The arithmetic unit stores data relating to the names and functions of the genes from which the nucleotide sequences of the DNA probes 8 in the respective regions on the DNA chip 51 are derived. It can estimate and output the features of the sample DNA based on the correlation between the data and the read results.

The fluorescent marker after hybridization is detected preferably by using a confocal microscope, for a high S/N ratio and an improved detection sensitivity. The DNA chips 51 are preferably scanned by a combination of the rotational movement and monoaxial linear movement of the DNA chips 51 with the fixation of the optical system, or by a combination of the rotational movement of the DNA chips 51 with the monoaxial movement of the optical system. This configuration significantly increases the scanning speed and shortens the read time of the chips as compared with regular biaxial scanning of an object in the X- and Y-directions on stage.

This configuration also enables easy carrying out of a reading method in which scanning is repeated only on a small number of focused probe sites without repeating scanning of all the sites, for example, when signals from the probe sites have low intensities, and the S/N ratio is improved by an integrating assay.

When reading at plural wavelengths must be conducted, the reader has only to further comprise additional components such as light sources and photoreceptors.

The reader preferably further comprises a focusing servo mechanism that serves to keep the distance between the lens and the circumferential side wall of the DNA chip 51 even when the surface level of the circumferential side wall of the DNA chip 51 varies with the rotation. This enables precise detection of the fluorescence even when the surface of the DNA chip 51 is uneven and the position (level) of the surface of the DNA chip varies with the rotation. The distance between the surface of the DNA chip 51 and the lens can be determined, for example, by an astigmatic method, a Foucault process, a knife edge method, or an SSD method.

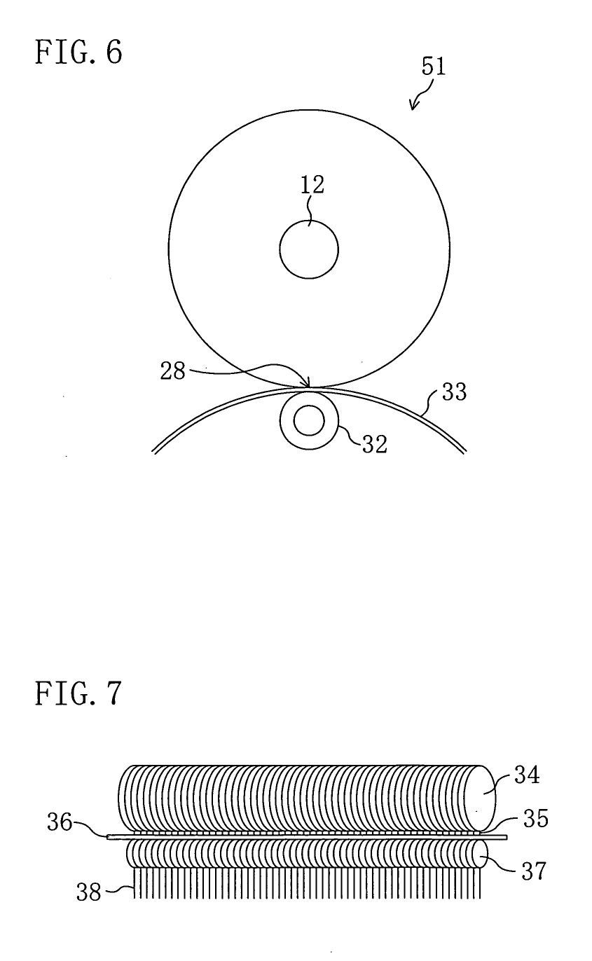

An example of the DNA chip reader is one comprising a read disc 32 and a membrane 33. The read disc 32 faces the discoidal DNA chip 51 and includes a measuring sensor, and the membrane 33 is arranged between the read disc 32 and the chip 51, as illustrated in FIG. 6. A probe on the DNA chip 51 is in contact with the measuring sensor in the read disc 32 on only one line on the circumference. Thus, the probe 28 at the specific position of the DNA chip 51 is assayed by the measuring sensor, and the specific position is determined by the position of the rotated disc or scrolling of the filter membrane 33.

With reference to FIG. 7, a discoidal chip assemblage 34 is treated with a carbon nanotube on its surface, is connected to contact terminals 35, and is subjected to hybridization with the sample. The discoidal chip assemblage 34 after hybridization is arranged so as to face a read disc 37 with the interposition of a membrane filter 36 intercalated with an organic light emitting material. The read disc 37 carries precisely arranged conductive interconnections 38. When a pulse voltage is applied from the conductive interconnections 38 to the discoidal chip assemblage 34, an electron flow efficiently flows from a hybridized probe via the carbon nanotube to thereby allow the membrane filter 36 to emit light. Consequently, a charge is accumulated in a CCD device integrated in the read disc 37 to thereby determine a DNA bound to the probe.

The hybridization reaction can be analyzed and evaluated by overlaying the membrane filter 36 on a film of a photosensitive material, and developing the film exposed to the emitted light. The membrane filter 36 can also be overlaid on an imaging plate. In this case, photons are accumulated in the imaging plate and are released upon analysis to thereby record the hybridization reaction. When a chemical light emission is carried out, the filter is combined with an enzymatically labeled antibody against a sample labeled with, for example, digoxigenin, is dipped in a substrate, is allowed to react, and the resulting light emission is determined.

### THIRD EMBODIMENT

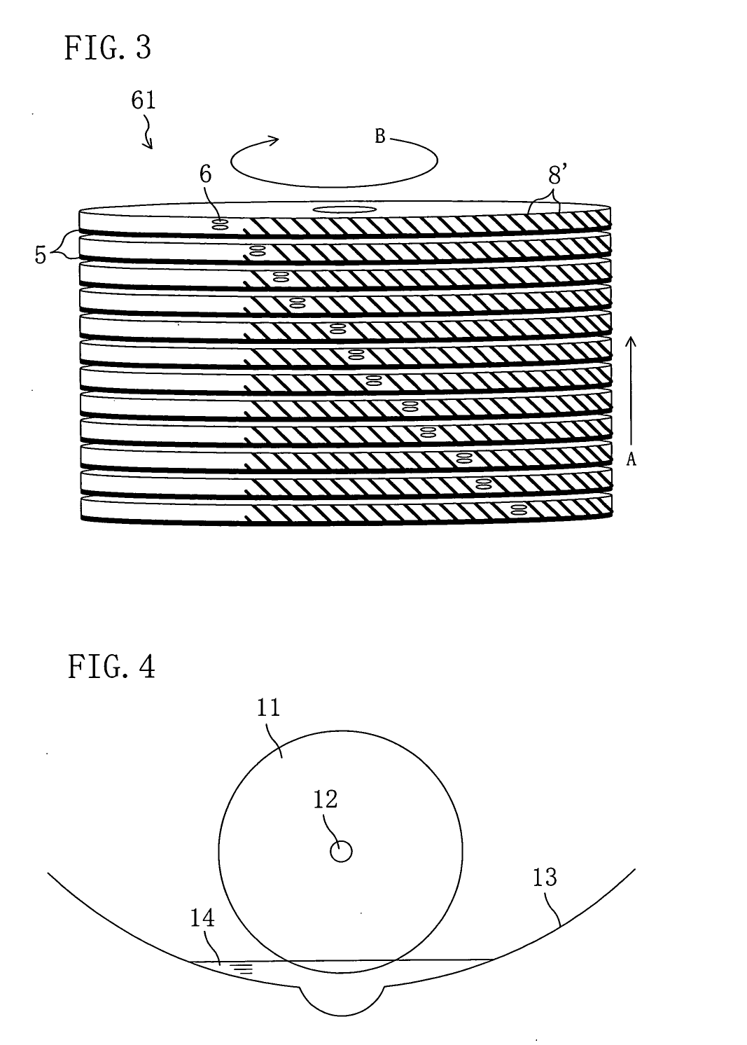

According to Third Embodiment as illustrated in FIG. 3, probes 8′, 8′ of plural types can be immobilized simultaneously by using a discoidal chip assemblage as in Second Embodiment and synchronizing the linear movement A in parallel with the central axis of the discs with the rotational movement B around the disc axis to as to keep the positional relation constant between all the probes 8′, 8′ groups and the index markers 6 in all the plural layers. The operation time can be significantly shortened according to this embodiment, for example, by using a unit having ten printer heads, preparing ten different solution of probes, and carrying out immobilization of ten different probes in one step. This technique requires only sixty printing operations for the total immobilization procedure, whereas a unit having a single printer head requires six-hundred printing operations.

For example, twenty discoidal DNA chips each carrying immobilized probe groups of six-hundred different types can be prepared in one process. In this case, twenty thin discs are sequentially stacked so as to have central axes coinciding with each other, as in Second Embodiment. A flexible spacer comprising a material different from that of the discs is preferably arranged between adjacent layers. The spacer acts to protect the discs carrying immobilized probes while enabling good adhesion between layers, and to improve the identifying capability of the probe-immobilized sites. For example, by stacking discs each 1 mm thick with the interposition of thin spacers each 0.1 mm thick, a DNA chip assemblage in the form of a thick disc having a thickness of 21.9 mm and a diameter of 1 cm and carrying probes of 12000 different types is prepared.

### FOURTH EMBODIMENT

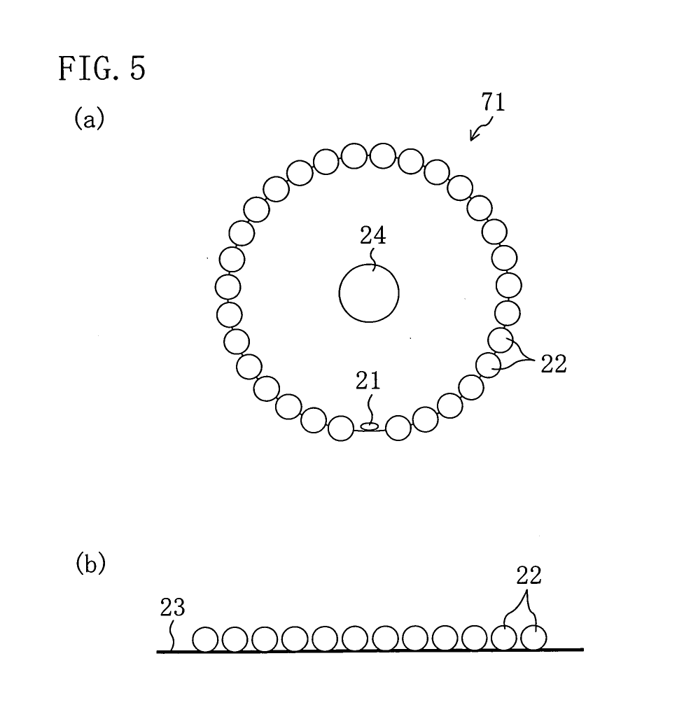

In a clinical blood biochemical testing system according to Fourth Embodiment, first assay reagents of thirty types are placed in predetermined reservoirs of ink-jet printers. The types of the first reagents can be selected from, for example, GOT, GPT, LDH, CPK, ALP, gamma-glutamyl transpeptidase, LAP, BUN, CRE, Ch-E, Na, K, Cl, Ca, P, Fe, Mg, ammonia, sialic acid, blood sugar (BS), HbA1C (hemoglobin A(1C)), amylase, bilirubin, total protein, albumin, uric acid, cholesterol, neutral lipid, HDL cholesterol, LDL, CRP, lipase, and various immunological items. A biological chip according to this embodiment is a discoidal chip. The chip has a similar shape to the DNA chip according to First Embodiment and further comprises incubation cells 22, 22 as containers on its circumferential side wall, as is schematically illustrated in FIG. 5(a). Each of the incubation cells 22, 22 has data of, for example, analyzed items, batch number, and patient identification number (ID) indicated by a two-dimensional barcode. Index marks 21 have a one-to-one correspondence with the contents and sequence (arrangement) of groups of the incubation cells 22, 22. Namely, a specific index mark 21 has a one-to-one correspondence with the contents and sequence (arrangement) of a specific group of the incubation cells 22, 22.

First reagents necessary for assay items predetermined on plural human plasma samples are discharged to the respective incubation cells 22, 22 by a ink-jet process in a manner corresponding to the sequence (arrangement) of the respective incubation cells 22, 22. The sequence herein has been stored by recognizing the index marks 21 of the biological chip 71. After one reagent is discharged, the biological chip 71 is rotated around the central axis in a spindle hole 24 by 10 degrees to thereby move a target incubation cell 22 to the position of a printer head from which another reagent is to be discharged, and the another assay reagent is discharged to the next incubation cell 22. This operation is repeated.

After all the reagents necessary for the predetermined items are placed in incubation cells, plasma samples are sequentially placed into the incubation cells 22, 22 containing the reagents while rotating the chip 71 by 10 degrees. In this procedure, blank incubation cell 22 is left without any treatment. For correcting the error in amounts, the amounts of samples used in the assay are corrected by adding a titration marker to all the samples and measuring the marker concentrations in diluted samples.

Thereafter, sufficient mixing typically by accelerated rotation is carried out, and an incubation for a suitable time is completed. If necessary, second reagents are placed into the incubation cells 22, 22, are mixed and are incubated in the same way as in the first reagents. After sufficient color development is achieved, the chip 71 is moved to an assay system by a simple vertical movement. The absorbance of the respective incubation cells 22, 22 are measured while rotating the chip 71 by 10 degrees. The assays of the items are conducted by calculation while referring to the stored sequence of the incubation cells 22, 22 and the origins of samples and comparing the data with standard curves. Finally, data are stored in patient files corresponding to the plasma samples and are outputted using a printer. Thus, the operation is completed.

For immunological assays, incubation cells carrying immobilized antibodies must be prepared. When detachable micro incubation cells 22 containing reagents in advance are used, incubation cells 22, 22 of thirty types corresponding to the predetermined assay items are taken from a shelf sequentially in accordance with a predetermined program so as to form a queue 23 as illustrated in FIG. 5(b). The queue 23 is then attached around the circumferential side wall of the chip 71. Next, plasma samples or diluted fluids thereof are placed into the incubation cells 22, 22 by an ink-jet process, and reactions at a constant temperature are carried out. Second reagents, if needed, are added, and the concentrations of absorbing substances formed in the incubation cells 22, 22 are measured, and measured data are evaluated by calculation. The sample data are summarized and are subjected to centralized control and output with the patient information.

The regular arrangement of incubation cells 22, 22 having a homogenous shape and a small capacity on the circumference of the chip as illustrated in FIG. 5 is useful for precisely and rapidly carrying out reactions and analyzing the results in a vary small system with a very small amount of sample, as compared with conventional techniques of injecting samples and reaction mixtures using an automatic sampler. For one-step reactions, reaction fluids (reactants) are preferably placed in incubation cells 22, 22 in advance. In contrast, two-step reactions can be proceeded, for example, by preparing dual-chamber incubation cells having a partition wall and breaking the partition wall. Alternatively, second reagents are placed in easily-breakable containers, and the reactions are proceeded by breaking the easily-breakable containers. Further alternatively, such second reagents are placed into incubation cells by an ink-jet process or using a micropipette.

Small amounts of fluids in the incubation cells 22, 22 can be effectively admixed by applying vibration generated by a vibrator or an acoustic wave or by centrifugal rotation. When the assay is conducted by absorptiometry (spectrophotometry) in the chip system, the assay can be carried, for example, by measuring light through walls of incubation cells made from a translucent material, by sampling the reaction mixture using a mini-sampler and measuring the absorbance thereof typically in a cuvette, or by inserting a microprobe for measuring the absorbance into the reaction mixture in the incubation cells 22. For precisely assaying local absorbance of fluids in such very small amounts, microphotometry, confocal analysis, or spectral analysis is preferably employed.

The reaction between a sample and, for example, a reaction reagent must be conducted in a liquid phase in the biological chip. However, a liquid reagent, if used, may evaporate or may decrease in its activity with elapse of time. To avoid these problems, the incubation cells preferably contain necessary reagents in the form of solids which have been prepared typically by freeze-drying. According to this configuration, reactions can be proceeded using reagents having precise concentrations and precise activities only by adding the sample or a diluted fluid thereof to the incubation cells. In this case, small amounts of fluids can be effectively admixed with powders by the action of vibration generated by a vibrator or an acoustic sound or by centrifugal rotation. Two-step reactions can be conducted according to this technique, for example, by adding a second reaction fluid upon the completion of the first reaction; by separating an incubation cell with a partition wall and breaking the partition wall upon initiation of the second reaction; or by arranging a reservoir for the second fluid, which reservoir can be broken typically by osmotic pressure or pressurization.

Solids such as tablets can be used as the first and second reagents, because such solid reagents can be easily placed into the incubation cells 22.

For carrying out an enzyme-linked immunosorbent assay (ELISA), a sample is preferably captured by sandwiching between two antibodies, for example, in the following manner. The sample is placed into an incubation cells bearing an immobilized first antibody to carry out a first reaction, the incubation cell is then rinsed, a liquid or solid second antibody is placed into the incubation cell to carry out a second binding reaction, the incubation cell is rinsed again, and the amount of the target molecule is determined using a light-emission or absorption reaction.

### OTHER EMBODIMENTS

For carrying out an ELISA, the biological chip according to any one of First to Fourth Embodiments is preferably used to thereby easily yield precise measured results in a short time. In a regular assay of an antigen molecule by ELISA, the antigen molecule having a known concentration must be measured simultaneously in every assay. Moreover, a lot of procedures, a lot of materials, and much efforts are required, for example, when 96-well plates are used. However, the biological chips according to the embodiments can solve the problems in conventional chips by incorporating the internal standard probe into the biological chips. Specifically, a probe reactive with a material (internal standard material) that is not contained in the sample to be measured is used as the internal standard probe; the internal standard material is added to a predetermined concentration to the sample; and a reaction between the biological chip and the sample is conducted. In this case, an ELISA can be easily, conveniently, and precisely conducted using only one biological chip carrying immobilized probes of plural types by employing a predetermined dissociation constant (Kd) of a light emitting or coloring reaction as a reference. In particular, two different internal standard materials and corresponding two different internal standard probes are preferably used. Thus, influences of the incubation environment and reaction inhibitory materials can be eliminated, and precise results can be easily obtained.

The above-mentioned embodiments are only examples of the present invention which by no means limit the scope of the present invention. The materials to be used as probes are not limited to DNAs and oligonucleotides and can also be any material selected from the group consisting of proteins, peptides, sugar chains, RNAs, lipids, cells, and fragments of these materials. The materials can also be artificial biologic materials such as synthetic DNAs.

Materials for the substrates of the biological chips include, in addition to polycarbonates, plastics such as polyamides and polyesters; glass; silicon; metals; ceramics; and composite materials of these materials.

The chip for use in the method for assaying a chip after the reaction with a sample with the interposition of, for example, a membrane or sheet between the chip and an assay terminal is not limited to a cylindrical or columnar chip carrying probes immobilized on its circumferential side wall. This method can also be applied to an assay using a conventional plate-like chip having probes as a matrix on its principal plane.

Of the biological chips according to the present invention, DNA chips are preferably used in combination with DNA chip readers. By using these, all the probes regarding a lot of domains of plural gene groups can be arranged in sections on stacked constitutional thin sheets of plural different types. Accordingly, arbitrary chips can be prepared in quantities at low cost by selecting constitutional thin sheets so as to cover focused groups of the whole gene groups. Alternatively, screening of a lot of gene groups can be carried out at random so as to reveal an unknown phenomenon. Thus, chips can be prepared and analyses can be conducted with high degrees of freedom. The chip can be scanned at high speed while ensuring a high S/N ratio using, for example, a confocal microscope by the combination of the linear movement of the chip and the disc rotation without deflections of the axis. In addition, the surface of the circumferential side wall is not immersed in a fluid (medium containing the sample) all the time, and the contact between the medium containing the sample and the probe during incubation is intermittent but stable and homogenous. Thus, precise determination can be achieved as a result of satisfactory hybridization, even when a small amount of the medium containing the sample is used. By using the chips for blood biochemical tests, assays that require much smaller spaces and are more useful for reducing cost and wastes than conventional equivalents can be established.

### INDUSTRIAL APPLICABILITY

As is described above, according to the biological chips of the present invention, probes or reactors of plural different types can be easily prepared on one chip in a short time, and reactions with samples and assays after the reactions can be easily conducted in a short time. Thus, the present invention is useful for analyses of gene functions and for detection/analyses typically of proteins.

1. A biological chip comprising a substrate; and a probe as a biologic material or artificial biologic material immobilized on a surface of the substrate,
wherein the substrate is in the form of a column or cylinder, and
wherein the probe comprises one selected from the group consisting of nucleic acids, peptides, saccharides, lipids, cells, and fragments of these materials, and wherein the probe is immobilized on a circumferential side wall of the substrate.

2. The biological chip according to claim 1, further comprising an index mark indicating the position of the immobilized probe on the surface of the substrate.

3. The biological chip according to claim 2,
wherein the index mark comprises plural index marks,
wherein the probe comprises plural probes immobilized at plural positions in the substrate,
wherein the plural positions of the immobilized probes are separated into plural groups, and
wherein the index marks are so arranged at predetermined positions on the substrate as to distinguish one group from another

4. The biological chip according to claim 1, wherein the probe comprises plural probes and includes at least one internal standard probe.

5. A biological chip assemblage comprising stacked plural plies of the biological chip according to claim 1,
wherein the biological chips are so stacked that the central axes of the columns or cylinders substantially coincide with each other.

6. The biological chip assemblage according to claim 5, wherein a spacer is arranged between adjacent two biological chips.

7. An incubator serving to bring a sample into contact with the biological chip according to claim 1, the sample being reactive with at least one of the probes, to thereby carry out a reaction between the sample and the at least one of the probes,
wherein the incubator is so configured as to keep the central axis of the column(s) or cylinder(s) of the biological chip or biological chip assemblage being substantially horizontal and to rotate the biological chip or biological chip assemblage around the central axis, and
wherein the incubator is so configured as to immerse a vertically lower part of the biological chip or biological chip assemblage in a medium containing the sample.

8. An assay device for determining at least one of physical properties, chemical properties, and biological properties of a molecule bound to the probe on the biological chip according to any one of claims 1 to 4 or the biological chip assemblage according to claim 1,
wherein the assay device comprises a measuring section and is so configured as to rotate the measuring section along the circumferential side wall of the column(s) or cylinder(s) around the central axis of the column(s) or cylinder(s).

9. An assay device for determining at least one of physical properties, chemical properties, and biological properties of a molecule bound to the probe on the biological chip according to any one of claims 1 to 4 or the biological chip assemblage according to claim 1,
wherein the assay device is so configured as to carry out an assay while rotating the biological chip or biological chip assemblage around the central axis of the column(s) or cylinder(s).

10. The assay device according to claim 8, comprising a measuring section, and further comprising a mediator between the measuring section and the biological chip or biological chip assemblage.

11. A biological chip comprising:
a columnar or cylindrical substrate; and
plural containers arranged on the circumference of the substrate.

12. The biological chip according to claim 11, wherein the containers are detachably arranged on the substrate.

13. The biological chip according to claim 11, wherein the containers contain assay reagents.

14. The biological chip according to claim 13, wherein the assay reagents are solid.

15. The biological chip according to claim 13, wherein the assay reagents are each a reagent which specifically reacts with a material to thereby determine the presence or absence of the material and to determine one of the quantity and activity of the material, wherein the material is selected from the group consisting of genes, and molecules including GOT, GPT, LDH, CPK, ALP, gamma-glutamyl transpeptidase, LAP, BUN, CRE, Ch-E, Na, K, Cl, Ca, P, Fe, Mg, ammonia, sialic acid, blood sugar (BS), HbA1C (hemoglobin A(1C)), amylase, bilirubin, total protein, albumin, uric acid, cholesterol, neutral lipid, HDL cholesterol, LDL, CRP, digoxin, theophylline, valproic acid, phenytoin, thyroid hormone, TSH, HBs (hepatitis B surface) antigen, HCV (hepatitis C virus) antibody, GH, LH, FSH, prolactin, ADH, ACTH, renin activity, aldosterone, myoglobin, ANP, BNP, erythropoietin, insulin, PSA, CEA, CA 19-9 (carbohydrate antigen), and AFP.

16. The biological chip according to claim 15, wherein at least one of the containers contains plural assay reagents of different types.

17. The biological chip according to claim 11, wherein the containers carry identifying marks on their outer surfaces for distinguishing one container from another.
